(19) United States
(12) Patent Application Publication
(10) Pub. No.: US 2024/0090870 A1
(43) Pub. Date: Mar. 21, 2024

(54) INCLINATION ANGLE CORRECTION FOR ULTRASOUND-BASED DIAPHRAGM THICKNESS MEASUREMENTS

(71) Applicant: KONINKLIJKE PHILIPS N.V., EINDHOVEN (NL)

(72) Inventors: Thomas Koehler, Hamburg (DE); Rafael Wiemker, Hamburg (DE); Comelis Petrus Hendriks, EINDHOVEN (NL); Michael Polkey, London (GB); Joerg Sabczynski, Hamburg (DE); Roberto Buizza, EINDHOVEN (NL); Jaap Roger Haartsen, EINDHOVEN (NL); Nataly Wieberneit, Hamburg (DE)

(21) Appl. No.: 18/210,672

(22) Filed: Jun. 16, 2023

Related U.S. Application Data
(60) Provisional application No. 63/407,769, filed on Sep. 19, 2022.

(51) Int. Cl. A61B 8/00; A61B 8/08; A61M 16/00; G06T 7/00

(52) U.S. Cl. CPC A61B 8/4254; A61B 8/4444; A61B 8/46; A61B 8/5207; A61B 8/54; A61M 16/022; G06T 7/0012; A61B 2560/02; A61B 2560/04; A61B 2562/0219

(57) ABSTRACT

A diaphragm imaging device includes at least one electronic processor programmed to perform a diaphragm imaging method including receiving ultrasound imaging data of a diaphragm of a patient, the ultrasound imaging data being acquired by an associated ultrasound imaging probe with the probe at a plurality of different observable probe angles (β_{obs}); for each observable probe angle, determining a corresponding apparent thickness (d_{I}) of the diaphragm of the patient from the received ultrasound data acquired at that observable probe angle; and estimating a thickness (d_{D}) of the diaphragm of the patient based at least on the apparent thicknesses (d_{I}).

### CROSS-REFERENCE TO RELATED APPLICATIONS

This patent application claims the priority benefit under 35 U.S.C. § 119(e) of U.S. Provisional Application No. 63/407,769, filed on Sep. 19, 2022, the contents of which are herein incorporated by reference.

The following relates generally to the respiratory therapy arts, mechanical ventilation arts, ventilator induced lung injury (VILI) arts, ultrasound probe arts, and related arts.

### BACKGROUND

Diaphragmatic ultrasonography (US) allows for quantification of diaphragm thickness, strain (rate) and excursion, and with this also the respiratory rate and duration of each contraction. Diaphragm thickness (expressed as thickening fraction) and strain reflect contractile activity and correlate well with diaphragmatic electrical activity and diaphragmatic pressure. Consequently, thickness and strain may be used as a surrogate for respiratory effort. Applications of diaphragmatic ultrasound include assessment of diaphragm function, atrophy detection, weaning prediction, and mechanical ventilation (MV) setting management. Other applications could be asynchrony detection and proportional ventilation (non-invasive neurally adjusted ventilatory assist (NAVA)). The use of diaphragmatic ultrasound in mechanical ventilation is gaining attention and therefore, technical problems and use cases are currently being investigated.

A diaphragm thickening fraction (TFdi or TFDI) as measured by ultrasound (US) are carried out by an operator who looks at the patient and takes an ultrasound image at end inhalation and end exhalation. The diaphragm thickness fraction is determined by subtracting the end inhalation thickness from the end exhalation thickness and dividing the difference by the exhale thickness according to Equation 1:

$$TFdi = \frac{{T}_{ei} - {T}_{ee}}{{T}_{ee}} * 100 \% \qquad ( 1 )$$

with Tₑᵢ as the end-inspiratory thickness. The thickness of the diaphragm as varying over the breathing cycle (i.e. the thickening during inspiration) is a surrogate for the patient's respiratory effort (see, e.g., Tuinman P R, Jonkman A H, Dres M, Shi Z H, Goligher E C, Goffi A, de Korte C, Demoule A, Heunks L. Respiratory muscle ultrasonography: methodology, basic and advanced principles and clinical applications in ICU and ED patients—a narrative review. Intensive Care Med. 2020 April; 46(4):594-605. doi: 10.1007/s00134-019-05892-8. Epub 2020 Jan. 14. PMID: 31938825; PMCID: PMC7103016).

Diaphragm thickness measurements can be obtained with ultrasound (US) imaging. However, such measurements can suffer from artificial thickening if the US beam does not pass the diaphragm perpendicularly. If the US beam has an inclination with respect to the surface normal of α, then the diaphragm thickness appears to be d/cos α, i.e., it appears to be thicker by a factor of 1/cos α than it actually is. This can lead to inaccurate diaphragm thickness measurements.

The following discloses certain improvements to overcome these problems and others.

### SUMMARY

In one aspect, a diaphragm imaging device includes at least one electronic processor programmed to perform a diaphragm imaging method including receiving ultrasound imaging data of a diaphragm of a patient, the ultrasound imaging data being acquired by an associated ultrasound imaging probe with the probe at a plurality of different observable probe angles (β_{obs}); for each observable probe angle, determining a corresponding apparent thickness (d_{I}) of the diaphragm of the patient from the received ultrasound data acquired at that observable probe angle; and estimating a thickness (d_{D}) of the diaphragm of the patient based at least on the apparent thicknesses (d_{I}).

In another aspect, a diaphragm imaging method includes, with at least one electronic controller, receiving ultrasound imaging data of a diaphragm of a patient, the ultrasound imaging data being acquired by an associated ultrasound imaging probe with the probe at a plurality of different observable probe angles (β_{obs}); for each observable probe angle, determining a corresponding apparent thickness (d_{I}) of the diaphragm of the patient from the received ultrasound data acquired at that observable probe angle; and estimating a thickness (d_{D}) of the diaphragm of the patient based at least on the apparent thicknesses (d_{I}).

One advantage resides in acquiring accurate diaphragm thickness measurements.

Another advantage resides in correcting an angle of an ultrasound probe that is imaging a diaphragm to obtain an accurate diaphragm thickness measurement.

Another advantage resides in providing feedback to a user to correct an inclination angle of an ultrasound probe while imaging a diaphragm.

Another advantage resides in correcting an artificial thickening factor in a diaphragm thickness measurement.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative diaphragm imaging device in accordance with the present disclosure.

FIG. 2 shows a different embodiments of a probe of the device of FIG. 1.

FIGS. 3-5 show example operations of the probe of FIG. 1.

FIG. 6 shows an example flow chart of operations suitably performed by the device of FIG. 1.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

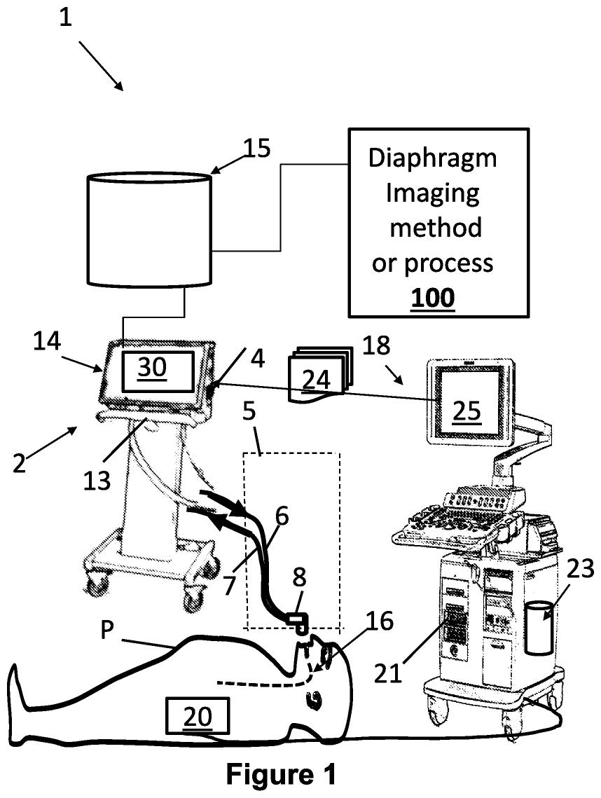

With reference to FIG. 1, a diaphragm imaging device 1 is shown. A mechanical ventilator 2 is configured to provide ventilation therapy to an associated patient P is shown. As shown in FIG. 1, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 also shows a medical imaging device 18 (also referred to as an image acquisition device, imaging device, and so forth). As primarily described herein, the medical imaging device 18 comprises an ultrasound (US) medical imaging device 18. The illustrative embodiments employ brightness mode (B-mode) ultrasound imaging to assess the diaphragm thickness metric. However, other types of ultrasound imaging or data are contemplated, such as motion mode (M-mode) data collected as a single ultrasound line over a time interval, or so forth.

In a more particular example, the medical imaging device 18 includes an ultrasound probe 20 that is configured to image the diaphragm of the patient P. The US probe 20 is positioned to acquire US imaging data (i.e., US images) 24 of the diaphragm of the patient P. For example, the US probe 20 is configured to acquire imaging data of a diaphragm of the patient P, and more particularly US imaging data related to a dimension (e.g., a position, a thickness, and so forth) of the diaphragm of a patient P during inspiration and expiration while the patient P undergoes mechanical ventilation therapy with the mechanical ventilator 2. In one example, the medical imaging device 18 includes an electronic processor 21 configured to control the ultrasound imaging device 18 to acquire the US images 24, and also includes a non-transitory computer readable medium 23 storing instructions executable by the electronic processor 21. The medical imaging device 18 can also include a display device 25. In another example, the electronic processor 13 of the mechanical ventilator 2 controls the ultrasound imaging device 18 to receive the ultrasound imaging data 24 of the diaphragm of the patient P from the US probe 20. The ultrasound probe 20 allows for continuous and automatic acquisition of the diaphragm thickness data (Tdi) from the acquired ultrasound imaging data 24.

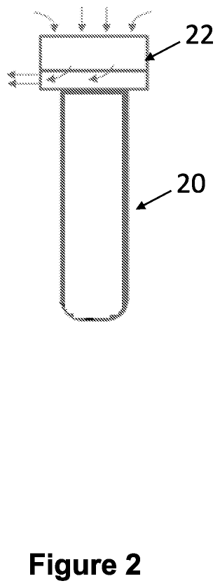

FIG. 2 shows an example of the ultrasound probe 20. An orientation of the probe 20 can be adjusted or moved relative to skin of the patient P in order to compensate for an inclination angle α of the probe 20 during imaging of the diaphragm of the patient P. To do so, the probe 20 includes an actuator 22 configured to move the ultrasound probe 20 relative to the skin of the patient P. The actuator 22 can comprise any suitable component, such as a thrust-producing device (i.e., a fan), a gyroscope, and so forth. The actuator 22 is configured to provide thrust (diagrammatically shown in FIG. 2 with arrows) in order to move (and correctly orientation) the probe 20 relative to the skin of the patient P during imaging of the diaphragm.

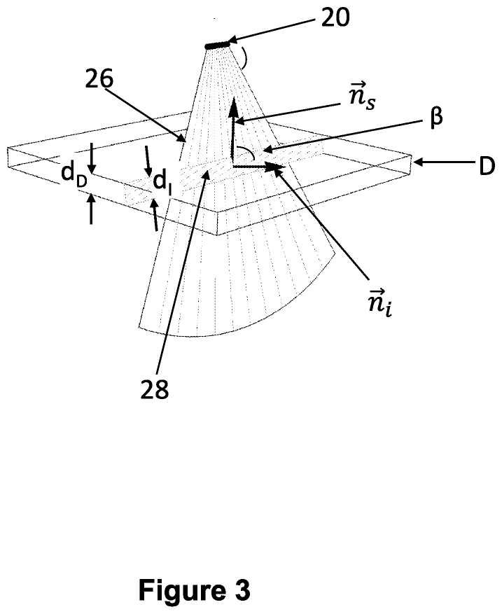

FIGS. 3-5 show example operations of the US probe 20. As shown in FIGS. 3-5, the ultrasound beam 26 is in the shape of a planar fan emitted from the ultrasound probe 20 towards a surface of a diaphragm D. The diaphragm D is approximately planar, and has a physical thickness d_{D} as indicated in FIGS. 3-5. This physical thickness d_{D} changes over the respiratory cycle, with the physical diaphragm thickness d_{D} being largest at end-inspiration due to contraction of the diaphragm muscle sheet, and with the physical diaphragm thickness d_{D} being smallest at end-expiration due to relaxation of the diaphragm. An intersection of the fan of US beams 26 and the diaphragm D is denoted in FIGS. 3-5 as diaphragm image or representation 28. A first normal vector \vec{n}ᵢ is the unit-length normal vector to the plane of the US fan beam 26. The normal vector \vec{n}ᵢ is thus also the unit-length normal vector to the plane of the image or representation 28 of the diaphragm D in the ultrasound image. A second normal vector \vec{n}ₛ is the unit-length normal vector to the surface of the physical diaphragm D, An angle β exists between the first and second normal vectors, and the angle β is defined as a scalar product of the vectors according to β=arccos \vec{n}ₛ⋅\vec{n}ᵢ where “⋅” denotes the dot product (i.e. scalar product). The physical thickness of the diaphragm D is denoted in FIGS. 3-5 as thickness d_{D}. However, an observed thickness d_{I} of the image 28 of the diaphragm D in the US imaging data 24 is broadened by a factor of 1/sin|β| where |⋅| denotes absolute value. In other words, the observed thickness d_{I} of the image 28 of the diaphragm D in the US imaging data 24 is:

$${d}_{I} = \frac{{d}_{D}}{sin \vert β \vert} .$$

Note that in the limiting case where the US fan beam 26 is oriented at 90° to the plane of the diaphragm D, then β=90° and sin(β)=1 and the imaged diaphragm thickness d_{I} is equal to the physical diaphragm thickness d_{D}, that is, d_{I}=d_{D}. However, obtaining this ideal perpendicular orientation is generally difficult or impossible using the external ultrasound probe 20 in an intercostal or subcostal position. Rather, the angle β will generally be less than (or greater than) 90° and hence d_{I}>d_{D} due to the tilt of the US beam fan 26 away from the perpendicular with the diaphragm D.

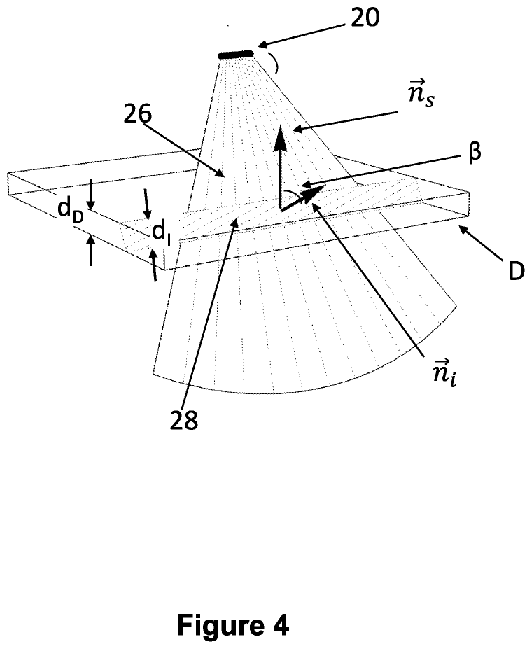

The US probe 20 can be moved by an operator relative to a skin surface of the patient P to acquire the US imaging data 24. If the fan of US beams 26 remains in a single plane, then the angle β does not change since the image plane does not change. Thus, the appearance of the thickness d_{I} of the image 28 of the diaphragm D does not change. On the other hand, if the US probe 20 is tilted so that the image plane changes, then the angle β changes. This is shown in FIG. 4, in which the US probe 20 is tilted as opposed to the position of the US probe 20 shown in FIG. 3. As shown in FIG. 4, when the US probe 20 is tilted relative to the surface of the diaphragm D, the orientation of the first unit normal vector \vec{n}ᵢ of the fan of US beams 26 changes, resulting in a change in the angle β. The thinnest appearance of the intersection 28 occurs when the angle β is 90°, but as previously noted this perpendicular orientation can be difficult or impossible to attain with a physical US probe. Moreover, since the diaphragm D is an internal organ, the angle β cannot be directly observed or measured.

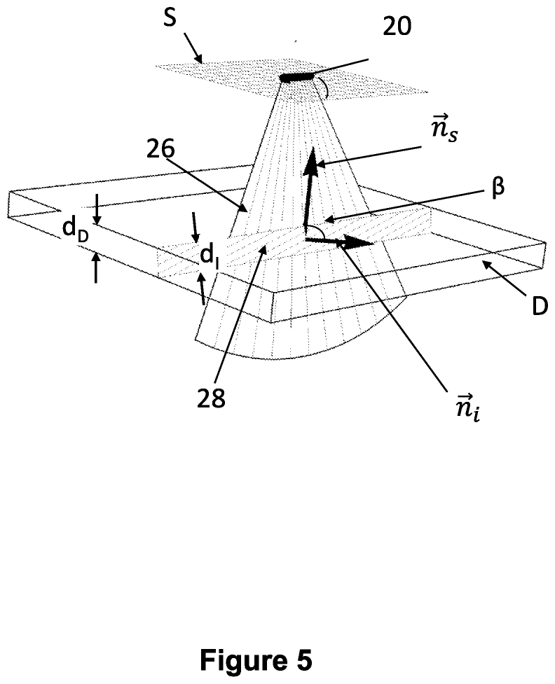

In some examples, one or more external sensors (e.g., a camera—not shown) can be used to determine and angle between the image plan of the fan of US beams 26 and a skin surface S, as shown in FIG. 5. Since the surface S of the skin is in general not parallel to the diaphragm D, the angle β cannot be directly inferred from an observation of the angle of the ultrasound probe 20 relative to the skin. However, a change in US probe orientation relative to the skin can be correlated with a change in the angle β between the plane of the US fan 26 and the plane of the diaphragm D. As the ultrasound acquisition frame rate is relatively fast (e.g. typically at least around 10 Hz or higher), by varying the angle of the ultrasound probe 20 with the skin the angle β can also be varied. In one approach, the apparent (i.e. imaged) diaphragm thickness

$${d}_{I} = \frac{{d}_{D}}{sin \vert β \vert}$$

is measured for several angles of the ultrasound probe 20 respective to the skin (and hence equivalently for several different values of the angle β) and the smallest value of the apparent (i.e. imaged) diaphragm thickness d_{I} is taken as being equal to the physical diaphragm thickness d_{D}. This estimated value of d_{D} may not be exact if the fan beam 26 cannot be positioned to be exactly perpendicular to the plane of the diaphragm D; however, since the derivative

$$\frac{d ( {d}_{I} )}{d β}$$

becomes small as β approaches 90° the estimated value of d_{D} may have sufficient accuracy. Note that this estimation of d_{D} is done for different points in the respiratory cycle, typically at least including end-inspiration (where d_{D} is thickest) and end-expiration (where d_{D} is thinnest).

If greater accuracy is desired, then the set of datapoints can be extrapolated based on the expectation that the d_{I} versus US probe angle curve should follow the expected

$$\frac{1}{sin β}$$

shape. For example, denoting the observable angle of the ultrasound probe 20 to the skin as an angle βₛₖᵢₙ, several ultrasound images can be acquired at different values of β_{obs} to produce a dataset of (d_{I}, β_{obs}) data pairs. The angle β is given as β=β_{obs}−β_{ref} where β_{ref} is an (unknown) angle offset between the unobservable angle β between the fan beam 26 and the plane of the diaphragm D and the observable angle β_{obs} between the fan beam 26 and the skin (or other observable US probe angle reference). Then the set of (d_{I}, β_{obs}) data points are fitted to the equation

$${d}_{I} = \frac{{d}_{D}}{sin \vert β \vert}$$

with the substitution β=β_{obs}−β_{ref} yielding

$${d}_{I} = \frac{{d}_{D}}{sin \vert {β}_{obs} - {β}_{ref} \vert}$$

with the two unknown fitted parameters being d_{D} and angle β_{ref}. To accommodate noise in the (d_{I}, β_{obs}) data points, they can be fitted to a sinusoidal curve which is then matched to

$${d}_{I} = \frac{{d}_{D}}{sin \vert {β}_{obs} - {β}_{ref} \vert} .$$

Again, this estimation of d_{D} is done for different points in the respiratory cycle, typically at least including end-inspiration (where d_{D} is thickest) and end-expiration (where d_{D} is thinnest). If images are acquired at 10 Hz or faster (i.e., at least 10 images per second) versus a respiratory cycle with a breath rate of typically no faster than about 60 breaths per minute (1 Hz) even for a newborn patient, sufficient data can be collected by sweeping the ultrasound probe 20 over a range of observable angles β_{obs} two or three times, e.g. using manual tilting of the US probe 20 with the observable angle Gobs monitored by an external sensor as described herein.

The non-transitory computer readable medium 15 of the mechanical ventilator 2 and/or the non-transitory computer readable medium 23 of the US imaging device 18 stores instructions executable by the electronic controller 13 (and/or the electronic processor 21) to perform a diaphragm imaging method or process 100. Although primarily described in terms of the electronic controller 13/non-transitory computer readable medium 15 of the mechanical ventilator 2, the method 100 can similarly be performed by the electronic processor 21/non-transitory computer readable medium 23 of the US imaging device 18.

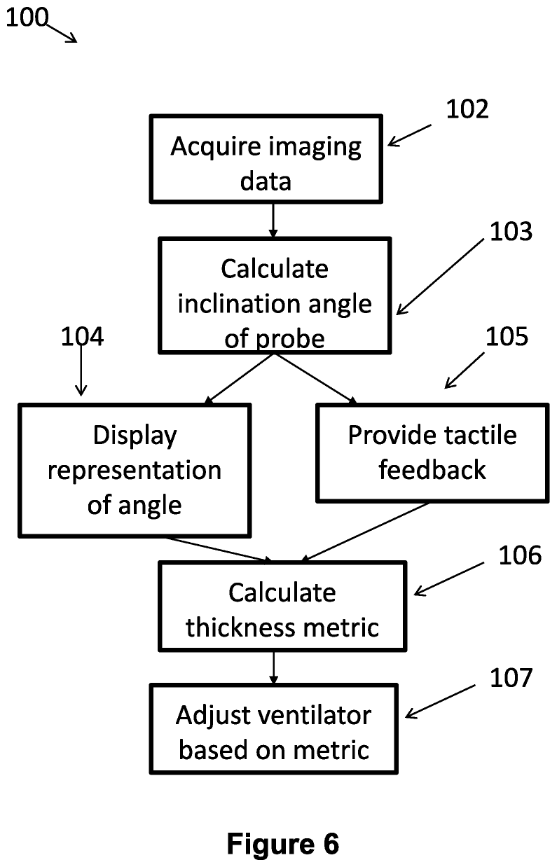

With reference to FIG. 6, and with continuing reference to FIGS. 1-5, an illustrative embodiment of the diaphragm imaging method 100 is diagrammatically shown as a flowchart. At an operation 102, the US imaging data 24 of the diaphragm of the patient P is acquired with the ultrasound probe 20 and transmitted to the electronic controller 13. In some embodiments, the US imaging data 24 is acquired while the patient P undergoes mechanical ventilation therapy with the mechanical ventilator 2. The US imaging data 24 includes data related to a geometry (e.g., position, thickness, etc.) of the diaphragm of the patient P. To do so, the electronic controller 13 can control the ultrasound probe 20 to acquire the ultrasound imaging data 24 and to receive the ultrasound imaging data 24 of the diaphragm of the patient P from the ultrasound probe 20. These images are not necessarily acquired while the patient P is on mechanical ventilation, but instead may be acquired (for example) prior to intubation of the patient.

At an operation 103, an inclination angle β of the ultrasound probe 20 is calculated from the US imaging data 24. To do so, a position of a surface of the diaphragm of the patient P is determined from US imaging data 24. For example, a clinician can acquire the US imaging data 24 at a plurality of different orientations (i.e., different angles) relative to the skin of the patient P. The position of the surface of the diaphragm can be determined from the US imaging data 24 acquired at the plurality of different orientations. From the determined position of the surface of the diaphragm, the inclination angle β of the probe 20 can be calculated.

Once the inclination angle β of the probe 20 is calculated, a corrective action can be performed. In some embodiments, at an operation 104, a representation 30 of the calculated inclination angle α can be displayed on the display device 14 of the mechanical ventilator 2 (or on the display device 25 of the medical imaging device 18). The operator of the probe 20 can then adjust the orientation of the probe 20 relative to the skin of the patient P until a desired inclination angle β of the probe 20 is achieved. In some examples, a representation 30 of the standard inclination angle β can be displayed on the display device 14, and a representation of the current inclination angle β of the probe 20 can also be displayed. The operator can then move the probe 20 until the current inclination angle β matches the standard inclination angle β.

In other embodiments, at an operation 105, tactile feedback can be provided for an operator of the probe 20. The tactile feedback can be provided by the actuator 22 of the probe 20 (e.g., the thrust-producing device or gyroscope can vibrate to indicate that the operator should change the orientation of the probe 20, automatically move the probe 20, and so forth). In a particular example, a difference between the calculated inclination angle β and a standard inclination angle β′ (i.e., the angle between the normal \vec{n}ᵢ of the image plane and the normal of the skin surface S) can be determined, and the tactile feedback can be provided until the calculated inclination angle β matches the angle β′.

At an operation 106, a diaphragm thickness metric (i.e., a thickness of the diaphragm or a diaphragm thickening fraction) can be calculated based on the US Imaging data 24 and/or the calculated inclination angle β. The displayed representation 30 can include a representation of the calculated diaphragm thickness metric. In one example, the diaphragm thickness metric includes a diaphragm thickening ratio indicative of a diaphragm thickness during inspiration relative to a diaphragm thickness during expiration. In another example, the diaphragm thickness metric includes a mean diaphragm thickness over multiple respiratory cycles.

At an operation 107, one or more parameters of the mechanical ventilation therapy delivered to the patient P by the mechanical ventilator 2 can be adjusted, for example, based on the calculated diaphragm thickness metric, the inclination angle β of the probe 20, and so forth.

In some embodiments, the actual inclination angle β can be estimated, and the operator can either wait for the correct inclination angle β of the US beam (i.e., during a manual sweep/attitude variation), or to correct for the artificial thickening (i.e., for a given attitude) caused by the inclination angle β of the probe 20. Since the US imaging data 24 is formed by a divergent set of US beams, the same geometric distortion of the diaphragm thickness appears also for the individual beams. However, the US imaging data 24 is commonly resampled to a Cartesian grid. Thus, the divergence of the beams is compensated, and the diaphragm appears in the image as two straight lines of distance d/sin|β|.

The operator continuously changes the inclination angle β of the probe 20 in a back-and-forth motion, which can be done at approximately 2 Hz (i.e., as shown in FIG. 4). The diaphragm thickness is continuously measured within the acquired US images 24 (i.e., as shown in FIG. 5), and the thinnest estimate is taken (because d/sin|β has a lower bound of d, i.e., the apparent thickness can never be shorter than the true thickness).

The operator continuously changes the inclination angle β of the probe 20 back-and-forth, and the diaphragm thickness is continuously measured. Additionally, an inclination angle γ of the US probe 20 with respect to the lab coordinate system is measured. This can be done, for instance, using accelerometers or magnetometers (which can measure the angle γ between the US probe 20 and the earth's magnetic field) integrated into the US probe 20 or by tracking the probe with external sensors (e. g. optically (i.e., using a camera) or via radiofrequency (RF) tags). The measurement provides pairs of data points (e.g., angle, thickness, etc.). A model of the apparent diaphragm thickness as a function of the actual thickness and the angle is fitted (where the model contains just the artificial thickening by 1/sin|β and the unknown angle between the diaphragm normal and the 0° angle in the lab coordinate system). The model parameter for the actual thickness is provided to the user. The benefit of this approach is that it takes all images from an ultrasound sweep with the probe 20, resulting in a robust estimate of the inclination angle β of the probe 20.

In an initialization phase of the method 100, a sweep of the US probe 20 across a broad range of inclination angles β can be performed, including tracking of 3D position and orientation of probe 20 (e. g. optically, via RF tags, and so forth) to generate US images. From these images, a position of a local diaphragm surface can be determined. During inhalation and exhalation by the patient P, the US images 24 of the diaphragm can be acquired with the probe 20. The inclination angle β of the probe 20 can be estimated with a surface model and the determined local diaphragm surface, and the orientation of the probe 20 can be corrected based on the estimated inclination angle β.

The tactile feedback operation 105 can be performed in a variety of manners. For example, the US probe 20 is brought into a correct orientation. Then, a gyroscope constituting the actuator 22 is activated, which naturally acts to maintain the probe 20 in its orientation. The gyroscope 22 may be integrated in the US probe 20 or mounted to the US probe 20 as an add-on. For example, to counteract an attitude variation of 5 rad/s with a torque of 0.1 Nm would require a gyroscope 22 with a 5 cm diameter flywheel of 150 g, spinning at 25 k RPM. Another approach is to stabilize the US probe 20 by the torque generated from a thrust produced by an axial or centrifugal fan provided as an additional component of the actuator 22. Again, such a thrust generating device 22 could be integrated in the US probe 20 or mounted to the US probe 20 as an add-on. For example, a thrust of about 1 N (i. e. a torque of about 0.1 Nm when applied at 10 cm from a rotation point) could be generated by a 7.5×7.5 cm 2 centrifugal fan (e. g. ebm-papst RL 48-19/14) discharging through a 5 mm diameter tube. The active feedback can also be used to ensure the optimal contact area between the US probe 20 and the patient P.

In another embodiment, a 3D US probe 20 is employed, or a US probe 20 with two orthogonal fans. The US imaging data 24 is converted into a 3D point cloud, into which two 3D planes are fitted in a robust fashion (using e.g., a RANSAC approach). Finally, the orthogonal plane distance is provided to the operator.

It is beneficial to synchronize the US imaging data 24 with the mechanical ventilator 2 (i. e., thickness measurements are taken automatically in time windows around maximum inspiration and maximum expiration). This synchronization can be done using a common clock. The synchronization between the US probe 20 and the mechanical ventilator 2 allows comparing a measurement from different breaths. If consecutive breaths have similar respiratory muscle activity, points in the respiratory cycle can be selected from multiple breaths in a way that the chosen measurement gives the thickening fraction with the minimum diaphragmatic thickness for every point, this could ensure that the correct inclination angle β of the US probe 20 was used. This process can be applied to a subcostal and an intercostal data acquisition scenario.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A diaphragm imaging device, comprising:
at least one electronic processor programmed to perform a diaphragm imaging method including:
receiving ultrasound imaging data of a diaphragm of a patient, the ultrasound imaging data being acquired by an associated ultrasound imaging probe with the probe at a plurality of different observable probe angles (β_{obs});
for each observable probe angle, determining a corresponding apparent thickness (d_{I}) of the diaphragm of the patient from the received ultrasound data acquired at that observable probe angle; and
estimating a thickness (d_{D}) of the diaphragm of the patient based at least on the apparent thicknesses (d_{I}).

2. The device of claim 1, wherein the estimating includes:
estimating the thickness (d_{D}) of the diaphragm of the patient as the smallest apparent thickness of the determined apparent thicknesses (d_{I}).

3. The device of claim 1, wherein the estimating includes:
fitting the data pairs each comprising one of the determined apparent thicknesses (d_{I}) and its corresponding observable probe angle (β_{obs}) to an expected relationship between apparent thickness and observable probe angle.

4. The device of claim 3, wherein an expected relationship between the corresponding apparent thickness (d_{I}) and each observable probe angle (β_{obs}) is
$${d}_{I} = \frac{{d}_{D}}{sin \vert {β}_{obs} - {β}_{ref} \vert}$$
and the unknown fitted parameters are thickness (d_{D}) of the diaphragm of the patient and a reference angle β_{ref}.

5. The device of claim 1, wherein the observable angle (β_{obs}) is measured during the receiving by:
determining a position of a surface of the diaphragm of the patient from ultrasound imaging data of the dimension of a diaphragm of a patient; and
calculating an inclination angle of the associated ultrasound imaging probe (20) from the determined position of the surface of the diaphragm.

6. The device of claim 5, wherein determining a position of a surface of the diaphragm of the patient from ultrasound imaging data of the dimension of a diaphragm of a patient includes:
acquiring ultrasound imaging data of the dimension of a diaphragm of a patient based on a plurality of different orientations of the associated ultrasound imaging device relative to skin of the patient;
determining the position of a surface of the diaphragm from the acquired ultrasound imaging data of the dimension of a diaphragm of a patient.

7. The device of claim 1, wherein the method further includes performing a corrective action based on the estimated thickness (d_{D}) of the diaphragm, the corrective action comprising:
displaying, on a display device, a representation of the calculated inclination angle.

8. The device of claim 1, wherein the method further includes performing a corrective action based on the estimated thickness (d_{D}) of the diaphragm, the corrective action comprising:
providing, via the associated ultrasound imaging device, tactile feedback for an operator of the associated ultrasound imaging device.

9. The device of claim 8, wherein the corrective action comprises:
determining a position of a surface of the diaphragm of the patient from ultrasound imaging data of the dimension of a diaphragm of a patient; and
determining a standard inclination angle of the associated ultrasound imaging device from the determined position of the surface of the diaphragm at which the associated ultrasound imaging device is able to image the surface of the diaphragm of the patient.

10. The device of claim 9, wherein the corrective action comprises:
determining a difference between the calculated inclination angle and the standard inclination angle; and
providing the tactile feedback until the calculated inclination angle matches the standard inclination angle.

11. The device of claim 1, further including:
an ultrasound imaging device comprising an ultrasound probe configured to acquire the ultrasound imaging data of the dimension of the diaphragm of the patient;
wherein the ultrasound probe includes an actuator disposed on a portion of the ultrasound probe and configured to move the ultrasound probe relative to skin of the patient.

12. The device of claim 1, wherein calculating an inclination angle of the associated ultrasound imaging device includes:
calculating the inclination angle using one or more sensors.

13. The device of claim 1, wherein the method further includes:
calculating a diaphragm thickness metric based on the received ultrasound imaging data of the diaphragm of the patient; and
displaying, on a display device, a representation of the calculated diaphragm thickness metric.

14. The device of claim 1, further including:
a mechanical ventilator configured to deliver mechanical ventilation therapy to the patient, wherein receiving the ultrasound imaging data of a dimension of a diaphragm of occurs during inspiration and expiration while the patient undergoes mechanical ventilation therapy with the mechanical ventilator; and the method further includes:
controlling an associated mechanical ventilator to adjust one or more parameters of the mechanical ventilation therapy delivered to the patient based on the calculated diaphragm thickness metric.

15. A diaphragm imaging method comprising, with at least one electronic controller:
receiving ultrasound imaging data of a diaphragm of a patient, the ultrasound imaging data being acquired by an associated ultrasound imaging probe with the probe at a plurality of different observable probe angles (β_{obs});
for each observable probe angle, determining a corresponding apparent thickness (d_{I}) of the diaphragm of the patient from the received ultrasound data acquired at that observable probe angle; and
estimating a thickness (d_{D}) of the diaphragm of the patient based at least on the apparent thicknesses (d_{I}).
